(19) United States
(12) Patent Application Publication
(10) Pub. No.: US 2018/0296784 A1
(43) Pub. Date: Oct. 18, 2018

(54) RESPIRATORY MASK ASSEMBLY FOR STABILIZING PATIENT INTERFACE

(71) Applicant: RESMED LIMITED, Bella Vista (AU)

(72) Inventors: Philip Rodney KWOK, Sydney (AU); Zoran MILIJASEVIC, Sydney (AU); Karthikeyan SELVARAJAN, Sydney (AU); Gregory Robert PEAKE, Sydney (AU)

(21) Appl. No.: 16/015,014

(22) Filed: Jun. 21, 2018

Related U.S. Application Data
(63) Continuation of application No. 14/800,135, filed on Jul. 15, 2015, now abandoned.
Application No. 14/800,135 is a division of application No. 12/081,114, filed on Apr. 10, 2008, now abandoned.
(60) Provisional application No. 60/907,609, filed on Apr. 11, 2007.

(51) Int. Cl. A61M 16/06; A61M 16/08

(52) U.S. Cl. CPC A61M 16/0622; A61M 16/0638; A61M 16/065; A61M 16/0825; A61M 16/0683; A61M 16/0666; A61M 16/06; A61M 2016/0661; A61M 2210/0618; A61M 2210/0625; A61M 16/0611

(57) ABSTRACT

A cushion for use with patient therapy may include a face contacting element having at least one portion including at least a first gel and a skin portion containing the first gel. The skin portion may be silicone based. The face contacting element may be substantially triangular, and at least one of the cheek regions and the lower region of the triangular face contacting element may include a concave section that curves inwardly towards the breathing chamber. The cushion may include first and second gels having one or more properties that are different from one another.

### CROSS-REFERENCE TO APPLICATION

This application is a continuation of U.S. application Ser. No. 14/800,135 filed on Jul. 15, 2015, which is a divisional of U.S. application Ser. No. 12/081,114 filed on Apr. 10, 2008, which claims priority to U.S. Provisional Application No. 60/907,609, filed Apr. 11, 2007, the entire contents of each of which are hereby incorporated by reference.

### BACKGROUND OF THE INVENTION

### 1. Field of the Invention

The present invention relates to the field of respiratory masks, e.g., for use with positive pressure therapy for patients who suffer from obstructive sleep apnea (OSA). In particular, the invention is directed to a mask assembly including a mask frame including at least one gel component, e.g., in the form of a cushion or forehead support.

### 2. Description of Related Art

Masks adapted for infusion of a fluid, e.g., gas, to a patient, in particular those suffering from OSA, are required to deliver the fluid, to seal on the patient's face, to be adaptable with any patient movement, and to be comfortable.

Some prior art masks may be comfortable and compliant, but do not seal optimally, thus making the masks less effective. Often, the frame for the mask is hard plastic and thus sealing and compliance must come from the facial cushion. Generally, this is not well done.

A very sensitive area of the face where seal is usually located is the nasal bridge region. Usually, any increase in pressure is directly translated to the nasal bridge region and is thus uncomfortable and even painful. Early gel masks had flowable-type gels which, when the membrane was worn out or ruptured, could leak out into the airways, which is an obvious health hazard. On the other hand, prior art mask systems available from DeVilbiss Serenity™ Gel, SleepNet IQ™, Respironics Profile Lite™ and Hans Rudolph Ultimate Seal™ include gels that are substantially not flowable.

Current gel masks do not seem to offer any great advantages with respect to sealing, except that patients may perceive them as intrinsically feeling that they would seal. Current sealing is actually done by a polyurethane membrane which does not last, stretches and/or corrugates, thus compromising the seal. An example of such a gel cushion is U.S. Pat. No. 5,884,624 to Barnett et al.

Gel is a good absorber of pressure (e.g., areas of high contact pressure may be redistributed), but not necessarily a good sealing medium, particularly when it has no compliance (e.g., by not being able to remain in intimate contact with the patient's skin due to minor relative movement, such as experienced by natural body movement). “Compliance” is the level of displacement achievable between the patient's face and cushion and/or the mask's ability to maintain a comfortable seal. ResMed's Activa™ cushion is an example of a cushion with very good compliance.

The lack of compliance and resilience may affect seal performance and may create localized pressure points such as on higher facial landmarks, especially the nasal bridge region.

Exemplary means to achieve good compliance in cushions includes Bubble® cushions that have very thin membranes able to utilize air pressure to maintain intimate contact with the patient's face.

More recent developments to improve compliance include ResMed's Mirage Activa™ mask that has a thin-walled, expandable cell between the cushion seal and the mask frame. The expandable cell maintains constant pressure of the seal onto the patient's face and allows for high levels of compliance to maintain seal even with large levels of mask instability relative to the patient.

DeVilbiss Serenity™ Gel includes a gel section that is not in contact with the patient's face; the thinner membrane is the seal.

Respironics Profile Lite™ has a deformable layer between the sealing region and the mask. This layer may somewhat pre-shape the cushion profile closer to an individual face, however has nearly no effect on improving compliance and seal performance. This somewhat customizable layer is required because the cushion does not have the ability to substantially redistribute contact pressure regions.

In summary, existing masks are lacking in two major areas: effective seal compliance and their ability to substantially redistribute contact pressure forces.

In fact, though constructed from known gel-like materials, prior art products behave and feel much like soft elastomers, rather than partly or substantially flowable fluids.

Also, compromises have been made to the softness (generally stiffer materials are used) so that structural integrity can be maintained. For example, a soft cushion may not seal well as the walls may splay outwards under pressure and leak.

A cushion that is made softer and made of softer materials, such as gel, may be better at redistributing forces on the face to eliminate contact pressure points and/or maximize the contact area to minimize contact pressure points (as smaller surface area results in higher pressure, i.e., Pressure=Force/Area).

The problem with constructing a softer cushion using current art methods is that they provide very limited structural integrity because the soft walls would splay outwards under pressure and cause leak. The construction methods are therefore limited in their “containment” ability.

### SUMMARY OF THE INVENTION

Accordingly, one aspect of the present invention is to address one or more problems associated with prior art gel systems.

An aspect of the invention relates to embodiments structured to create a substantially softer and therefore more comfortable gel mask that has structural integrity.

Another aspect of the invention relates to embodiments structured to provide containment of a gel seal to prevent the gel seal or side-walls from buckling or splaying outwards under treatment pressure or high headgear strap tension (causing mask to crush harder into the patient's face).

Another aspect of the invention is to provide a gel contained within a skin portion that is flexible and stretchable, e.g., elastic.

Yet another aspect is directed to a gel configuration that allows for the use of multiple gels or fluids with different properties to affect seal performance and compliance.

Still another aspect relates to a mask system having a gel that is substantially flowable but not pourable.

According to one embodiment of the invention, there is provided a cushion comprising a face contacting element having at least one portion including at least a first gel (e.g., flowable but not pourable); and a silicone-based skin portion (e.g., elastic, stretchable and flexible) containing at least the first gel.

According to another embodiment of the invention, there is provided a cushion comprising a face contacting element having at least one portion including at least a first gel and a second gel; and a skin portion containing at least the first and second gels, wherein the second gel is different from the first gel in at least one respect.

According to yet embodiment of the invention, there is provided a cushion comprising a face contacting element having at least one portion including at least a first gel, the face contacting element being substantially triangular and including a nasal bridge region, cheek regions and a lower region opposite to the nasal bridge region, said face-contacting portion defining at least in part a breathing chamber; and a skin portion containing at least the first gel, wherein at least one of the cheek regions and the lower region includes a concave section that curves inwardly towards the breathing chamber.

Another aspect of the invention relates to a cushion that provides adequate compliance without substantially increasing the size.

Another aspect of the invention relates to a cushion including at least a gel and at least one other fluid, e.g., gel layer and gas/air layer.

Another aspect of the invention relates to a cushion or seal including one or more of the following features: an elastic skin (improve flow of gel and comfort), flowable gel (comfort), elastic/spring cuff (increased comfort and acknowledges more sensitive regions around nose), variable spring rates around nose (acknowledges more sensitive regions around nose), bilobular design (to assist with seal containment), biased lean (to assist with seal containment), and seal support (to assist with seal containment). The above-noted features may be used independently or may be used in combinations.

Another aspect of the invention relates to a cushion including a face contacting element having at least a portion containing a gel and a containment wall provided adjacent the portion containing the gel. The containment wall is adapted to contain displacement of the portion in use.

Another aspect of the invention relates to a nasal prong arrangement including a base, a pair of nasal prongs provided to the base, and a deforming mechanism provided adjacent each nasal prong. Each of the nasal prongs contains a gel material adapted to deform and seal to the patient's nare. Each deforming mechanism is adapted to selectively engage and deform the respective nasal prong.

Another aspect of the invention relates to a nasal prong arrangement including a base, a pair of nasal prongs provided to the base, and a ring containing a gel material provided to each nasal prong. Each gel ring is adapted to be supported by a rib or base of the prong and provide a gel-type seal with the patient's nare in use.

Another aspect of the invention relates to a cushion including a gel component adapted to provide a seal and a deforming mechanism provided adjacent the gel component and adapted to selectively engage and deform the gel component.

Another aspect of the invention relates to a nasal prong assembly including a nasal prong arrangement and headgear adapted to support the nasal prong arrangement on the patient's face. The headgear includes a support strap containing a gel material that is adapted to sit over the patient's nose in use.

Another aspect of the invention relates to a cushion including a seal portion adapted to provide a seal with patient's face and a gusset portion extending from the seal portion and adapted to dampen movement of the cushion in use. The seal portion or gusset portion includes at least a portion containing a gel.

Other aspects, features, and advantages of this invention will become apparent from the following detailed description when taken in conjunction with the accompanying drawings, which are a part of this disclosure and which illustrate, by way of example, principles of this invention.

### BRIEF DESCRIPTION OF THE DRAWINGS

The accompanying drawings facilitate an understanding of the various embodiments of this invention. In such drawings:

FIG. 1 is a side view of a partial mask and cushion assembly according to a first embodiment of the present invention;

FIG. 2 is a front or patient side view of the partial mask and cushion assembly shown in FIG. 1;

FIG. 3A is a schematic plan view of a cushion according to an embodiment of the present invention;

FIG. 3B is a schematic plan view of a cushion according to yet another embodiment of the present invention;

FIG. 4A is a schematic view of a gel cushion including a variable profile according to an embodiment of the present invention;

FIG. 4B is a schematic view of a gel cushion including a containment wall according to an embodiment of the present invention;

FIG. 4C is a perspective view of gel cushion and its X-plane, Y-plane, and Z-plane according to an embodiment of the present invention;

FIG. 4D is a front view of a gel cushion illustrating lateral support provided by a containment wall according to an embodiment of the present invention;

FIGS. 5-7 are cross-sectional views of cushions according to embodiments of the present invention;

FIG. 8 is a partial cross-sectional view of a mask and cushion assembly according to an embodiment of the present invention;

FIG. 9 is an enlarged partial view of the mask and cushion assembly shown in FIG. 8;

FIG. 10 is a partial cross-sectional view of a mask and cushion assembly according to an embodiment of the present invention;

FIG. 11 is a partial schematic cross-sectional view of a mask and cushion assembly according to yet another embodiment of the present invention;

FIG. 12 is a partial cross-sectional view of a mask and cushion assembly according to still another embodiment of the present invention;

FIG. 13 is an enlarged view of the cushion and a portion of the frame shown in FIG. 12;

FIG. 14 is a partial cross-sectional view showing a mask and cushion assembly according to another embodiment of the present invention;

FIG. 15 is a partial cross-sectional view according to a mask and cushion assembly according to still another aspect of the present invention;

FIG. 16A illustrates a known nasal prong arrangement including silicone-type nasal prongs;

FIG. 16B illustrates a gel-type nasal prong and deforming mechanism according to an embodiment of the present invention;

FIG. 17 illustrates a nasal prong arrangement including gel rings according to an embodiment of the present invention;

FIGS. 18A-18B illustrate a gel component and deforming mechanism according to an embodiment of the present invention;

FIG. 19 illustrates displacement forces applied by one or more deforming mechanisms according to an embodiment of the present invention;

FIGS. 20A-20B illustrate a gel mouth seal according to an embodiment of the present invention;

FIG. 21 illustrates a gel mouth seal according to another embodiment of the present invention;

FIG. 22 illustrates headgear including a gel support strap according to an embodiment of the present invention;

FIG. 23 illustrates a nasal mask including gel pads according to an embodiment of the present invention;

FIG. 24 illustrates headgear including gel earplugs according to an embodiment of the present invention;

FIG. 25 illustrates a nasal prong arrangement including a support pad according to an embodiment of the present invention;

FIG. 26 illustrates headgear including gel and/or foam inserts according to an embodiment of the present invention;

FIGS. 27A-27E illustrate a mask including a cushion with a gel-filled gusset portion or gel-filled seal portion according to an embodiment of the present invention;

FIG. 28 illustrates headgear including gel and/or foam according to an embodiment of the present invention;

FIG. 29 illustrates a mask including a headgear clip with gel and/or foam pads according to an embodiment of the present invention;

FIG. 30 illustrates headgear including gel and/or foam ear pads according to an embodiment of the present invention; and

FIG. 31 illustrates headgear including a gel mattress according to an embodiment of the present invention.

### DETAILED DESCRIPTION OF ILLUSTRATED EMBODIMENTS

1. Cushion with Gel Layer

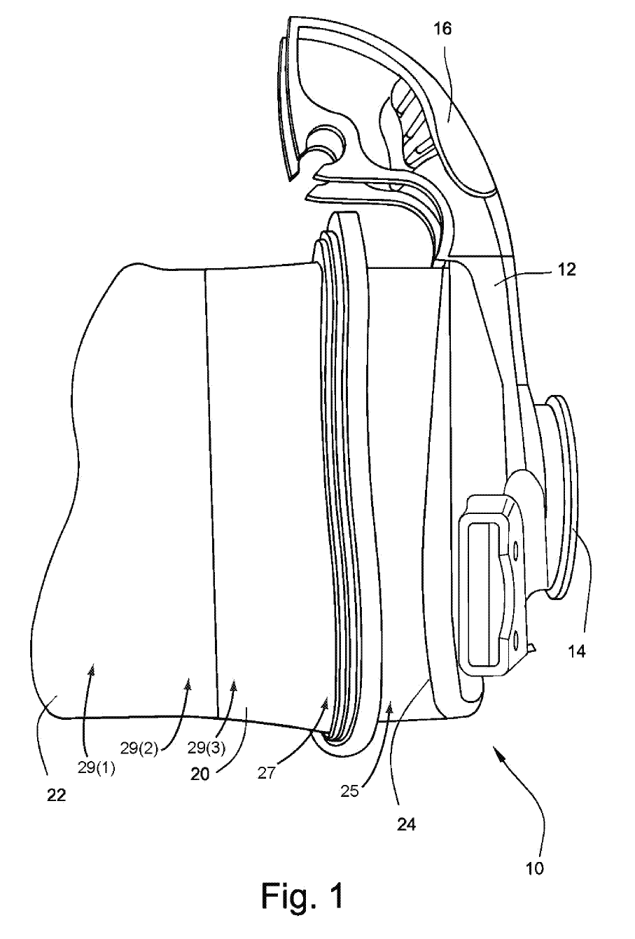

FIG. 1 is a side view of a mask assembly 10 according to a first embodiment of the present invention. Mask assembly 10 includes a frame 12 including an air delivery port 14 and a joining member 16 that is adapted to support an adjustable forehead support as shown, for example, in U.S. Pat. No. 6,532,961, incorporated herein by reference in its entirety.

A cushion 20 includes a face contacting element 22 and a frame engaging portion 24. An adaptor plate 25 is provided to fix the cushion 20 to the frame 12 and a cushion-to-frame attachment point 27, e.g., glue line, is provided to secure the cushion 20 to the frame 12. In the illustrated embodiment, the cushion 20 provides three layers, i.e., the first layer is a gel layer 29(1), the second layer is a separation layer 29(2), and the third layer is an elastic cuff or spring layer 29(3).

While the mask assembly shown in FIG. 1 is a nasal mask, the description that follows also has equal applicability to oro-nasal or full face masks as well. Further, while the face contacting element is described in conjunction with nasal or full face cushions, prongs, mouth seals, etc., the contacting element may also be in the form of a forehead support containing a gel component or pad having one or more of the features described below. Gel components may also be included with other parts of the mask assembly, such as head straps of headgear, etc.

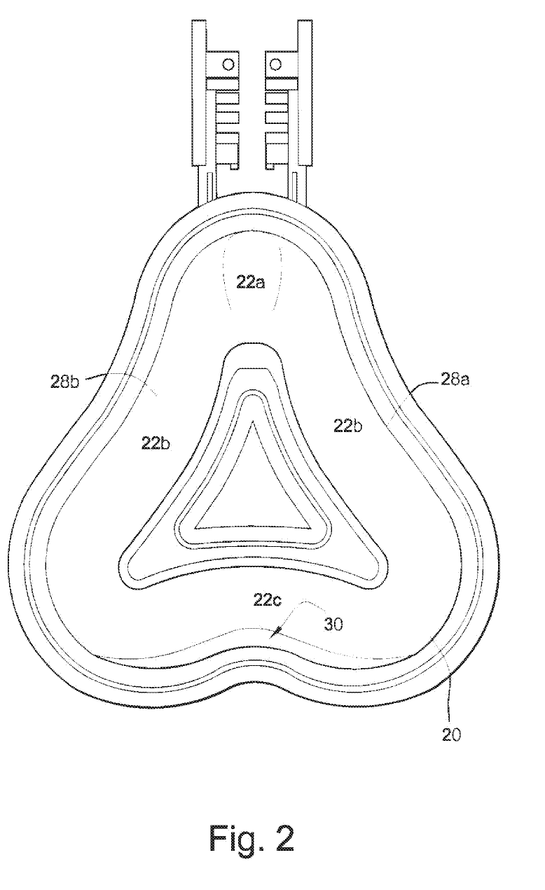

As shown in FIG. 2, the face contacting element 22 has a generally triangular shape defining a nasal bridge region 22a, cheek regions 22b and a lower region 22c which is adapted to contact either the patient's upper lip or lower lip/chin region depending on whether it is a nasal mask or an oro-nasal mask.

1.1 Concave Sections

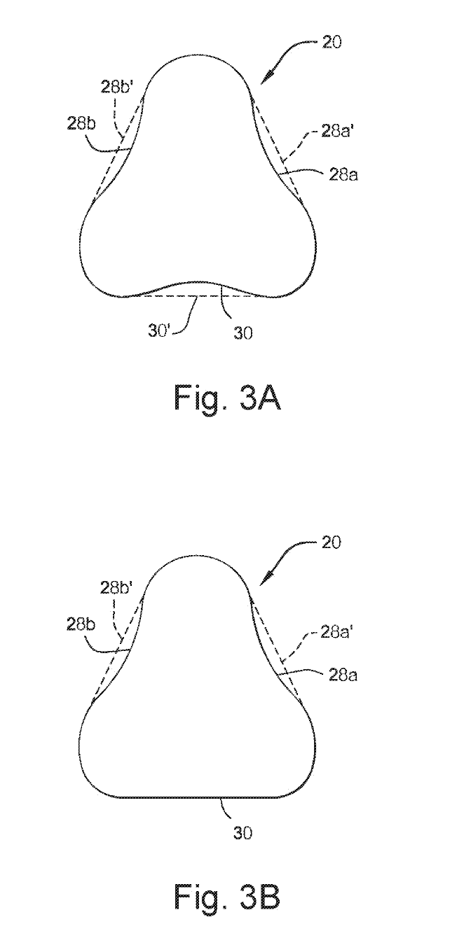

As shown in FIGS. 2 and 3A, each cheek region defines a concave section 28a, 28b, while the lower region 22c defines a concave section 30. The concave sections have a radius of curvature of about 180 mm, for example. As shown in FIG. 3A, each of the concave sections 28a, 28b and 30 can move from the solid line profile to the dotted line profile 28a′, 28b′ and 30′, upon application of fitting or strap pressure. The concave sections deform into a desirably substantially traditional triangular shape when fitted to the face. This is advantageous because the sides bulge outwards less than traditional masks, which minimizes leaks and also minimizes pressure in the eyes' tear duct region. A further advantage is a reduced risk of contact with the eye region that can cause patient discomfort and/or visual obstruction. However, the curvature may include a compound curve made up of more than one radius or include a completely free-from curve.

As shown in FIG. 3B, another embodiment of the invention envisions a cushion that includes concave sections 28a and 28b, but the lower lip region 22c includes a substantially linear profile, i.e., without a concave section.

2. Gel Seal or Cushion

2.1 Variable Profile

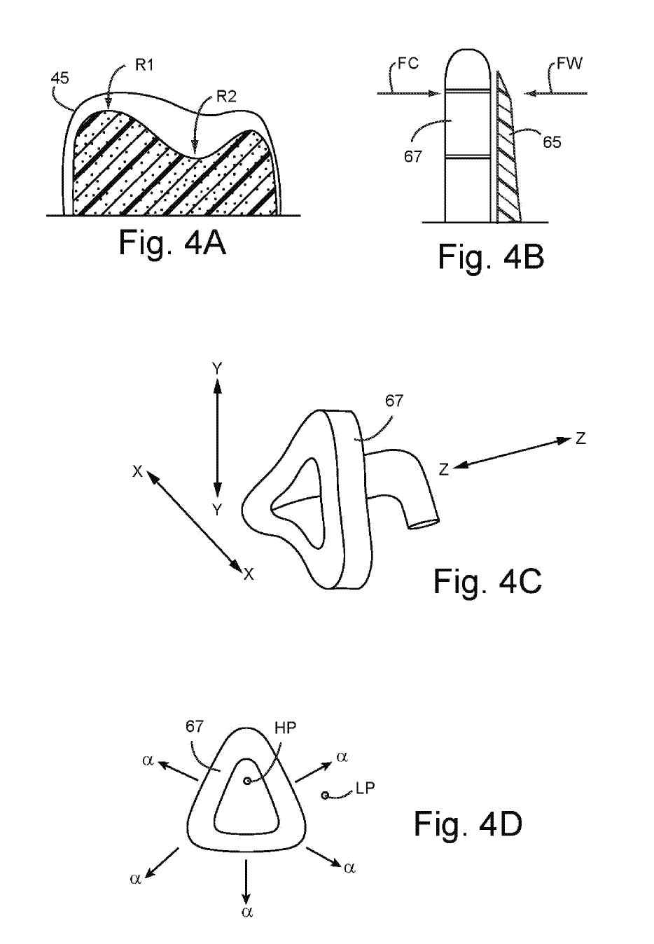

In an embodiment, the gel cushion may provide a variable profile/height around the gel cushion and flexibility may be modified by wall thickness or by using different thickness composites. For example, FIG. 4A illustrates a gel cushion 45 providing a variable profile in which more support is provided in a first region R1 and less support is provided in a second region R2. As illustrated, the second region R2 has a lower height that the first region R1.

2.2 Containment Wall

In an embodiment, a containment wall may be provided to the gel cushion to provide lateral stability and support. For example, FIG. 4B illustrates a flexible support or containment wall 65 that is structured to provide an opposing force FW to the general force FC provided by the gel cushion 67 (e.g., due to pressure in mask). The containment wall 65 may be retro-fittable or even integrated to the gel cushion, e.g., a flexible skeleton within the gel walls. Exemplary materials of the containment wall 65 include polyurethane, relatively stiffer silicone, and polypropylene.

FIG. 4C shows an exemplary gel cushion 67 and its X-plane (left/right displacement), Y-plane (up/down displacement), and Z-plane (in/out or towards/away from face). In an embodiment, the containment wall 65 may not affect the displacement and cushion compliance in the Z-plane.

FIG. 4D is a front view of an exemplary gel cushion 67 (e.g., cushion as patient views the mask) with higher pressure HP inside the mask and lower pressure LP outside the mask in surrounding atmosphere. In an embodiment, the containment wall may be structured to only counter the gel seal wanting to move in an outwards direction shown by arrows marked “α” in FIG. 4D, especially under influence of pressure. The outward movement of the seal may also be contributed by strap tension pulling the mask into the patient's face. The containment wall therefore provides lateral support to the seal to prevent leaks especially under the influence of pressure.

Testing shows that very little force may be provided to improve the seal by containment of the sealing region, e.g., see FIG. 1, element 22 and FIG. 4B.

The containment wall allows the cushion to remain soft and compliant for maximum comfort without compromising seal (e.g., prior art requires relatively stiffer cushion sidewalls to prevent leak).

Such embodiments of a gel seal or cushion may be applied into forehead pads or other interfacing pads, e.g., cheek pads.

3. Dual Wall Concept

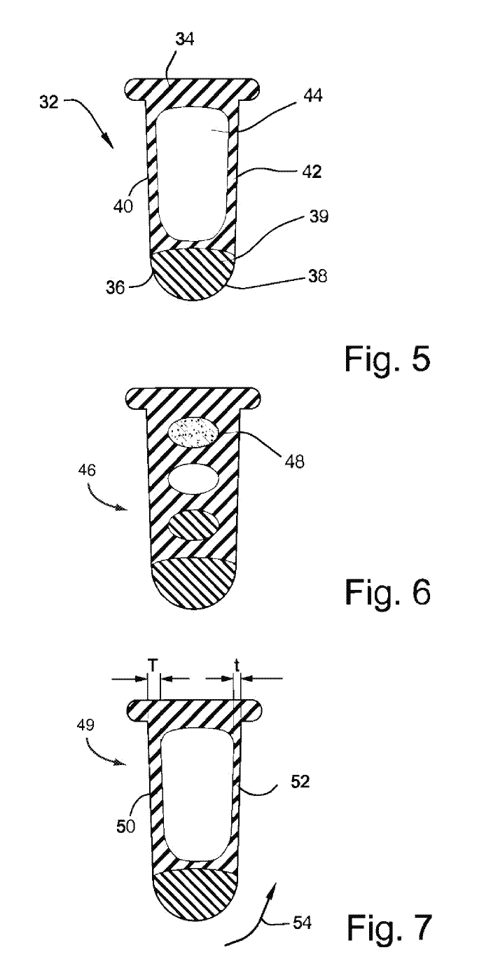

FIGS. 5-7 illustrate schematic cross-sections of cushions including face contacting elements according to embodiments of the present invention. The face contacting element may take the form of a substantially triangular shaped member, i.e., a face cushion, or it may be a forehead cushion for a forehead support.

FIG. 5 illustrates a face contacting element 32 including a base 34 and a tip 36 including a skin portion 38 that encases or contains a gel 39. Face contacting element 32 also includes an outer wall 40 and an inner wall 42 that are formed in a one piece integral unit from silicone in a preferred embodiment. A closed pocket 44 which may serve as a gas spring is defined between base 34, tip 36 and walls 40 and 42.

The dual wall construction shown in FIG. 5 provides a stiffer structure which will deform to provide larger conformance to the individual facial profile when additional strap tension is applied. The dual wall may take various forms, and the central air cushion may be formed from gas/air, gel, foam or any other suitable spring structure. For example, FIG. 6 shows a face contacting element 46 that includes up to three closed pockets 48 that may be filled with gel, foam and/or gas/air.

3.1 Roll Effect

FIG. 7 illustrates an embodiment of a face contacting element 49 including a relatively thick outer wall 50 and a relatively thin inner wall 52. Upon application of fitting or strap pressure, face contacting element 49 tends to roll in the direction of arrow 54 towards the nose or inside of the face due to the variance between the thickness of the inner and outer walls. The outer wall is approximately two to five times as thick as the inner wall. For example, the thickness T of outer wall 50 may be about 3 mm and the thickness t of inner wall 52 may be about 1 mm. In another embodiment, a part, parts, or the whole of the perimeter of the face contacting element 49 may be configured to roll outwardly.

3.2 Variations Around Perimeter

FIGS. 5-7 show a cross-section of one position along the perimeter of the face contacting element. It should be noted that variations of the structure and/or performance characteristics of the face contacting element around the perimeter are envisioned since different requirements for the face contacting element can be found around the perimeter of the mask. For example, the nasal bridge region is sensitive to excess pressure and requires a good seal to prevent air escaping and leaking into the eyes. The cheek regions at the sides of the mask are less sensitive and can help support the cushion structure. To provide the optimum cushion profile for these variations around the perimeter, the structure of the outer gel, the closed pocket and/or the structure of the dual wall and spring can be varied. For example, in the nasal bridge, the dual wall spring force may be designed to be small or non-existent and thus deformation can easily occur without excessively loading this sensitive region. The spring force can then be increased at the cheek regions to provide sufficient support for the cushion and mask structure.

3.3 Skin Portion Characteristics

Skin portion 38 is flexible and stretchable (e.g., elastic). This allows for compliance and adaptability of the mask to the user's face, similar to that of the ACTIVA mask, but without the extra section that makes the mask bulkier. This will happen without having any extra pressure as the gel will be allowed to conform with applied force, minimizing the resultant pressure. This is particularly true if used in conjunction with straps attached to the head only.

The skin portion containing the first gel may be elastic. The skin portion may be similar to the outer membranes of the cushion of ResMed's commercially available Ultra-Mirage® Full Face Mask which is made from silicone such as, e.g., Silastic®.

4. Gel Characteristics

The gel may be flowable but not pourable. An example of such a gel is commercially available from NuSil, a U.S. company, under the name Gel 8150. In addition, the gel may be dynamic and/or adaptive. Further, the gel is configured for temporary deforming or self-leveling.

Moreover, the gel and/or the skin portion can initially be placed under pretension, which leads to less crinkling of the skin portion when placed on the face and deformed further. The gel could have phase change properties that change with force, pressure or temperature.

5. Ball and Socket

FIG. 8 illustrates a partial cross-sectional view of a mask and cushion assembly with a face contacting element according to another embodiment of the present invention. Mask 51 includes a frame 53 including an air delivery tube 55 which delivers breathable gas under pressure to a breathing chamber 57 which is in communication with the patient's airways. Frame 53 includes a gas washout vent 56 for the continuous washout of exhaled gas from the patient.

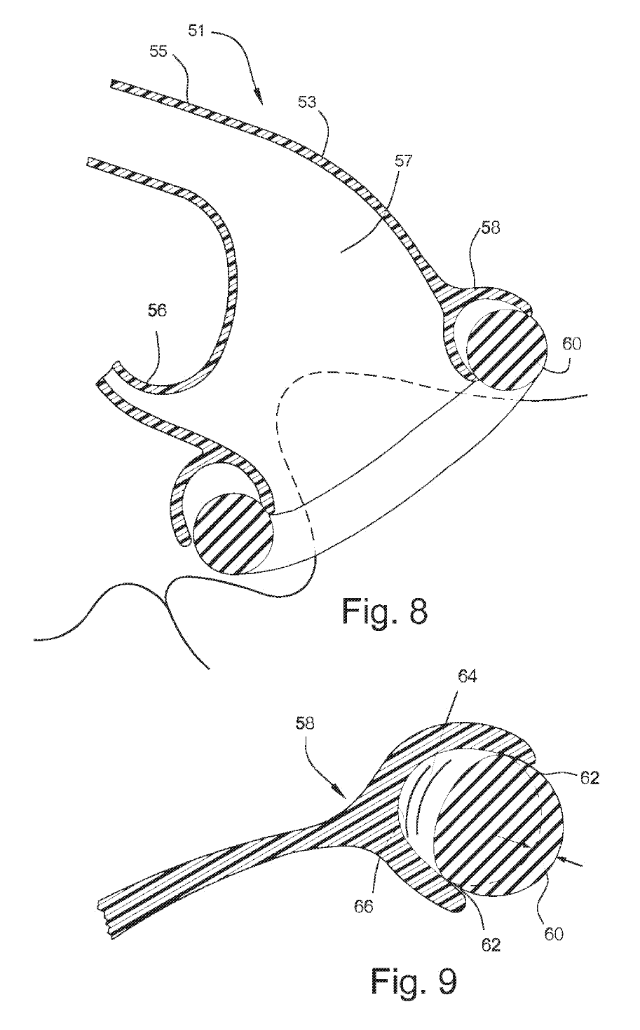

Frame 53 includes a cradle 58 which is constructed to support a face contacting element 60. As shown in the enlarged partial view in FIG. 9, cradle 58 includes open ends 62 forming a socket adapted to receive (e.g., fix or seal) the face contacting element 60 in the desired position, similar to a ball and socket joint. Preferably, an open pocket 64 is provided between the face contacting element and a base 66 of the cradle. The open pocket is substantially crescent shaped in the embodiment of FIGS. 8 and 9. The open pocket allows room for the gel of the face contacting element to expand, thereby allowing a better sealing fit with the patient.

The frame is made of a light polymer. The cradle is generally U-shaped in the embodiments of FIGS. 8 and 9.

6. Single Piece Mask

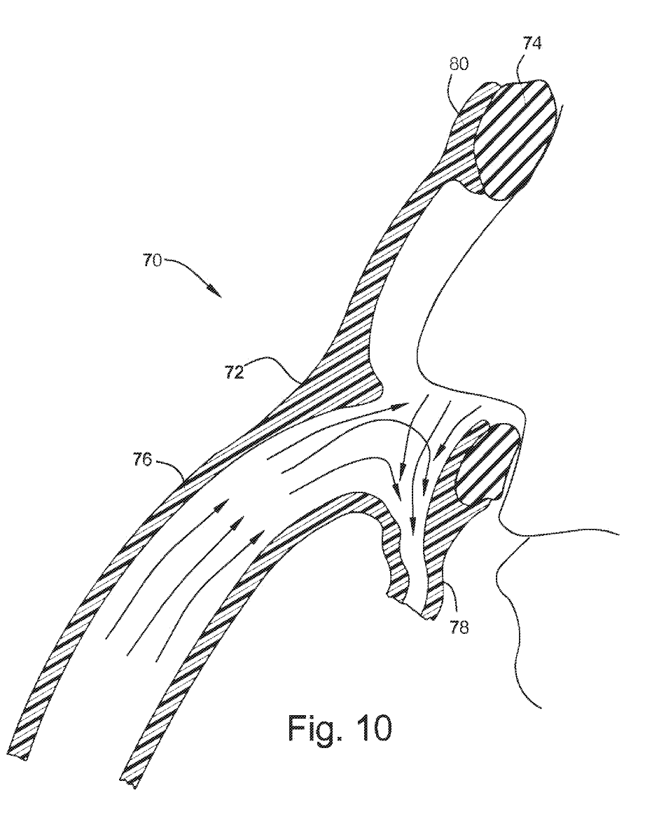

FIG. 10 illustrates yet another embodiment of the present invention. Mask assembly 70 includes a mask frame 72 which supports a face contacting element 74 having a gel component. Frame 72 defines an air delivery conduit 76 as well as a vent aperture 78. Frame 72 also defines a crescent shaped cradle portion 80 which is adapted to support face contacting portion 74. Air delivery conduit 76, vent aperture 78 and cradle portion 80 are designed as an integral one piece unit made from an elastomer.

7. Multiple Gel Mask

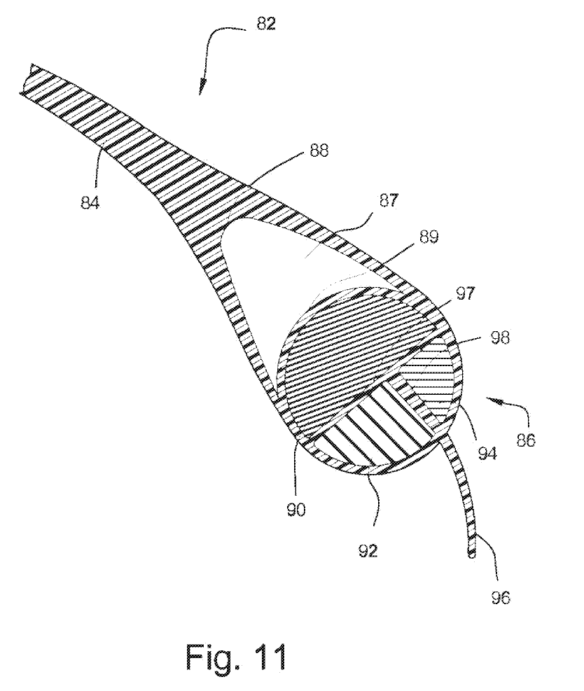

FIG. 11 illustrates yet another embodiment of the present invention. The portion of mask assembly 82 shown in FIG. 11 includes a frame 84 and a face contacting element 86. An arrow or parabola shaped pocket 87 is formed between a base portion 88 of frame 84 and a bottom end 89 of face contacting element 86.

Face contacting element 86 includes a skin portion which encapsulates or encases a first gel 90, a second gel 92 and a third gel 94, each of which may have properties that are different from one another. For example, gel 90 may be a relatively harder gel, while gels 92 and 94 may be relatively softer gels. The soft or resilient gels 92, 94 are used to seal and comply dynamically while the harder gel 90 is used to comply with the facial topography and any other slow changing external forces. Gels 90, 92 and 94 may be provided within separate and discrete chambers defined, for example, by divider walls 97, 98. In another variant, gels 92, 94 may be the same and/or have the same characteristics, while divider wall 98 may simply form a support for enhanced positioning and/or sealing.

A thin membrane 96 may be provided to extend away from the skin portion. The membrane 96 may extend outwardly in a direction that generally bisects the face contacting element. Membrane 96 may extend along a line that is aligned with divider wall 98 between second and third chambers containing the second and third gels 92, 94 respectively. Membrane 96 is structured to define a convex sealing surface projecting inside the face contacting portion.

8. Flat Cushion With Bellows

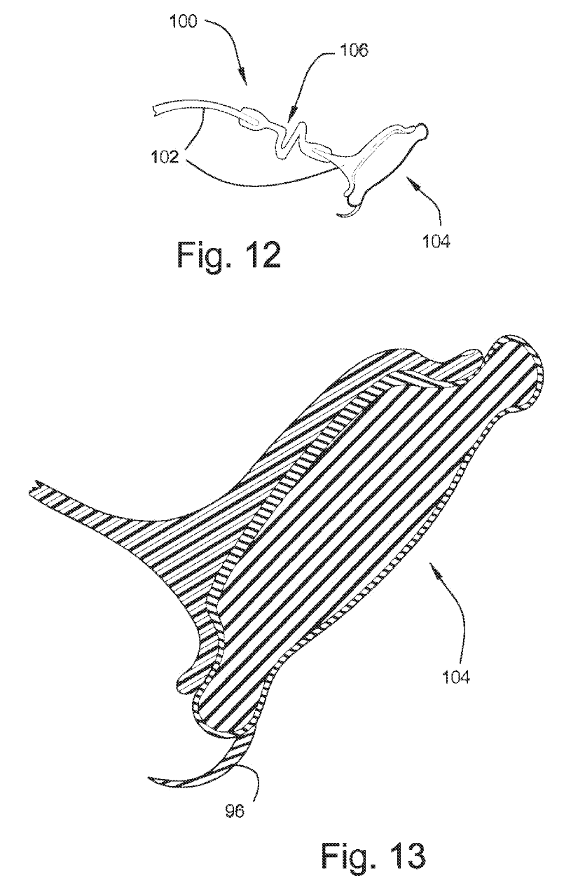

FIG. 12 illustrates a partial cross-sectional view of a mask assembly 100 according to yet another embodiment of the present invention. Assembly 100 includes a frame 102 and a face contacting element 104. A bellows 106 may be provided between frame 102 and face contacting element 104, for improved compliance. While the elastic bellows 106 is shown to include two female portions which receive corresponding male portions of the frame 102 in the face contacting element 104, the frame 102 may include a female connector while the bellows may include a male connector. In this way, the male portion of face contacting element can interact with either the female portion of the frame, or the female portion of the bellows 106.

FIG. 13 is an enlarged view of the face contacting element 104 shown in FIG. 12. Face contacting element may be generally flat and include one or more gels as described above. The face contacting element 104 may include a thin membrane 96 as described above.

9. Triple Gel Cushion

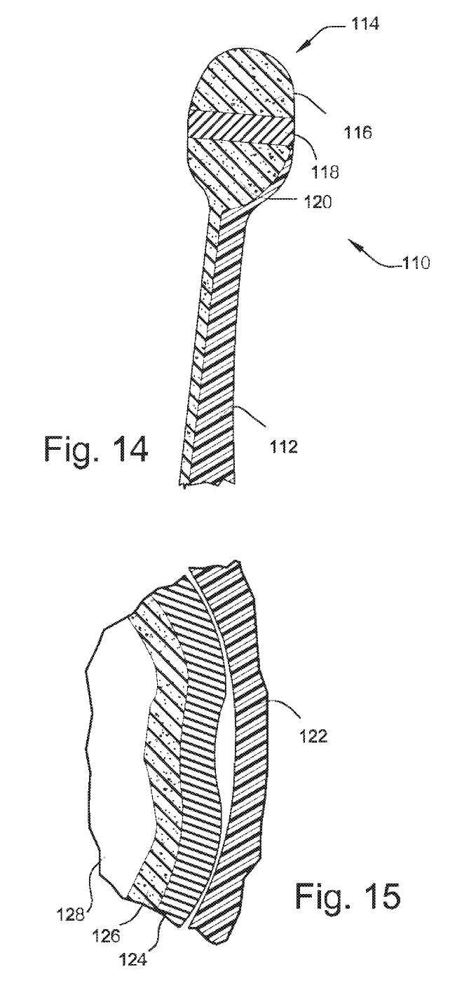

FIG. 14 is a cross-sectional view of a partial mask section 110 according to yet another embodiment of the present invention. Mask section 110 includes a frame 112 and a face contacting element 114. Face contacting element 114 may include first, second and third chambers or portions 116, 118, 120. The first and third chambers 116, 120 may include a resilient gel while the second chamber 118 may include a conforming gel positioned between the two resilient gel portions. The third gel 120 may extend along at least a portion of the frame 112, which extends along the inside of the mask to define at least a portion of the breathing chamber.

The use of multiple gels (two or more) provides three functions, sealing, compliance and comfort. The resilient gel can seal well and comply to any dynamic changes of the face or external disturbances of the mask. Most current masks do not seal well and comply with changes without high strap tension, which is not comfortable.

A slow flowing gel can adapt to the contours of the face and to any slow changing external disturbances. Other methods of adapting to facial contours include malleable wires and heat forming cushions have not proven to be successful and only allow for initial placement but they do not adapt to any changes and are not able to cope with other problems. By its very nature, gel is a soft comfortable substance. When a softer than human facial fat tissue gel is chosen, and combined with a stretchable skin, the cushion feels very comfortable under various conditions.

10. Dual Gel Cushion

FIG. 15 illustrates yet another embodiment of the present invention. FIG. 15 illustrates a partial mask assembly including a frame 122, and a face contacting element including a first cushion layer 124 and a second cushion layer 126 adjacent to a patient's face 128. First layer 124 may include a resilient gel, while second layer 126 may include a conforming gel.

11. Alternative Gel Applications

11.1 Seal

11.1.1 Gel Seal in Nostrils

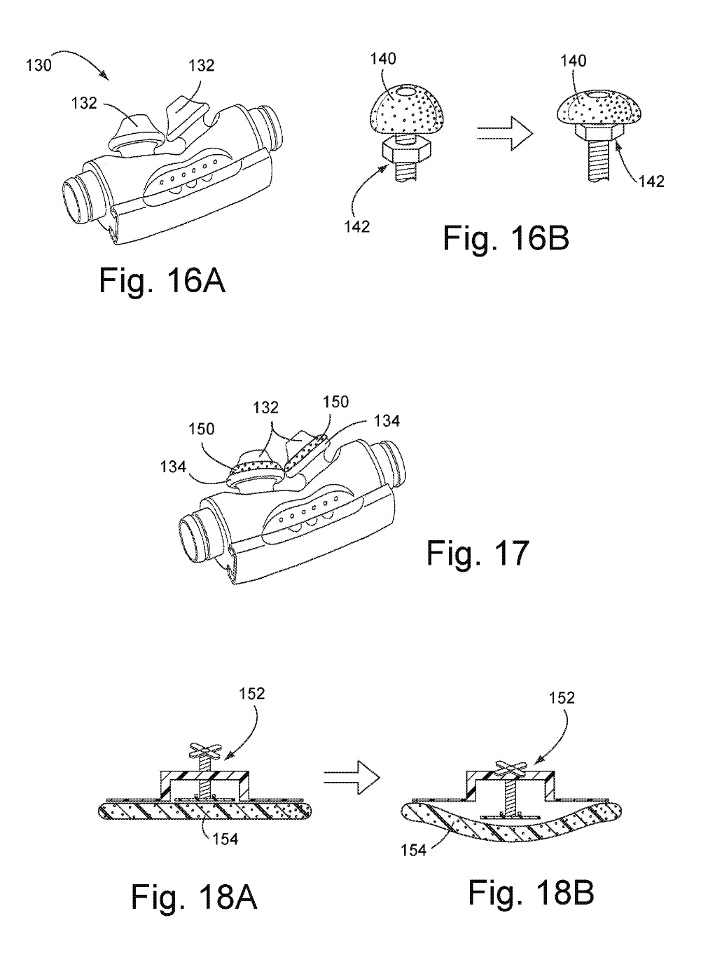

In an alternative embodiment, a gel-type seal may be provided to a nasal prong arrangement. For example, FIG. 16A illustrates a known nasal prong arrangement 130 including silicone-type nasal prongs 132. Such a nasal prong arrangement is commercially sold under the name of SWIFT® by ResMed Ltd., and further details and embodiments of such nasal prong arrangement are disclosed in U.S. Patent Publication Nos. 2004/0226566, published Nov. 18, 2004, and 2005/0241644, published Nov. 3, 2005, each of which is incorporated herein by reference in its entirety. In an exemplary embodiment, the silicone-type nasal prongs 132 may be replaced with gel-type nasal prongs such as the gel-type nasal prong 140 shown in FIG. 16B.

As shown in FIG. 16B, each gel-type nasal prong 140 may be formed of a gel material (e.g., gel-filled body) that is adjustable or deformable to perfectly fit and seal to the patient's nares (e.g., self-sealing “plug”). As illustrated, a deforming mechanism 142 (e.g., screw, cam, or pneumatic arrangement) may be provided adjacent the nasal prong 140, e.g., along a stalk portion, that is adapted to engage the prong 140 and force the prong 140 to bulge outwardly so that it conforms and seals with the respective nare. That is, the deforming mechanism 142 is adapted to change the diameter of the gel-type prong 140 by deforming the outer area of the gel-type prong 140. In use, the patient may adjust the amount of gel deformation 140 by selectively adjusting the deforming mechanism 142 to perfectly fit and seal the prong 140 to the respective nare.

In an embodiment, the gel-type prongs may be provided at the end of a flexible tube (e.g., if the seal is adapted to support the weight of the air delivery tube and prevent the air delivery tube from pulling the prongs out of the patient's nose).

In alternative embodiments, the deformable and self-sealing gel prongs may have other suitable forms or arrangements. Besides gel-filled bodies deformed by mechanical action, the prongs may include self-fitting or expanding bodies using expansion foam (e.g., similar to existing earplugs) or a combination of visco-elastic foam and gel (e.g., using the foam for the self-expanding fit and the gel as the seal against the inside of the patient's nares).

11.1.2 Visual Indicator Particles

In an embodiment, visual indicator particles (e.g., glitter) may be added to the gel material. The density of the particles changes as they are spread under contact pressure. In such an arrangement, the areas of highest deformation would become visually clear as the particles would spread apart from one another. The areas of highest deformation may indicate where pressure sores may become a problem. Once the problem areas are identified, they can be accommodated by the design of the cushion.

11.1.3 Gel Rings

In an embodiment, rings formed of a gel material may be provided to respective nasal prongs in an existing nasal prong arrangement (e.g., such as the SWIFT® nasal prong arrangement described above) to aid sealing. For example, as shown in FIG. 17, each gel ring 150 may sit on top of a respective prong 132, e.g., gel ring 150 supported by a rib or base 134 of the prong 132. However, each gel ring may be provided to a respective prong in other suitable manners, e.g., attached with an adhesive, integral one-piece, etc. Thus, each gel ring 150 is positioned between the silicone prong 132 and the patient's skin in use. The gel ring 150 provides a relatively sticky interface to provide a relatively sticky gel seal in use (e.g., similar to Hans Rudolph Ultimate Seal).

11.2 Seal Enhancement

11.2.1 Gel to Provide Even, Distributed Load

In an embodiment, gel may be used in a mask to turn or convert an adjustable point force into an evenly, distributed load. Specifically, mask seal may be improved or enhanced by using the deformable property of gel and its ability to adapt to the geometry of shapes.

A deforming mechanism may be provided to mechanically adjust the gel seal's shape, e.g., screws, cams, or other mechanical arrangements. The deforming mechanism along with the gel component, e.g., gel strip, cooperate to provide a seal. For example, FIGS. 18A and 18B illustrate a mechanical arrangement 152, e.g., screw adjustor, adapted to mechanically displace a gel strip 154 from a first configuration (FIG. 18A) to a second configuration (FIG. 18B) to provide a seal with a relatively smooth geometry and even, distributed load. In an alternative embodiment, the deforming mechanism may be in the form of a deformable metal band that may be deformed in a suitable manner, e.g., by hand, to mechanically adjust the gel seal's shape.

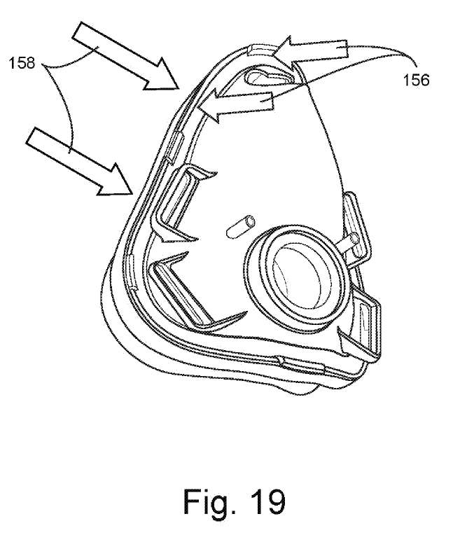

If sufficient deforming mechanisms (e.g., screw adjustors) are provided to a mask, this type of system may be used to significantly adjust the shape of the mask and provide a customizable fit. For example, as shown in FIG. 19, displacement forces may be applied by one or more deforming mechanisms to the gel component in a direction normal to the front plane of the mask (as indicated by the arrows 156) and/or displacement forces may be applied by one or more deforming mechanisms to the gel component in a direction parallel to the front plane of the mask (as indicated by the arrows 158).

11.2.2 Gel Pads

In an embodiment, gel pads may be used to “shape” the patient's nose to dilate nasal passages for a better shape for sealing.

11.2.3 Adjustable Force to Gel Pocket

In an embodiment, an adjustable force may be used to gel pocket to modify the shape and “hardness” in different regions of the mask.

11.2.4 Gel Mouth Seal

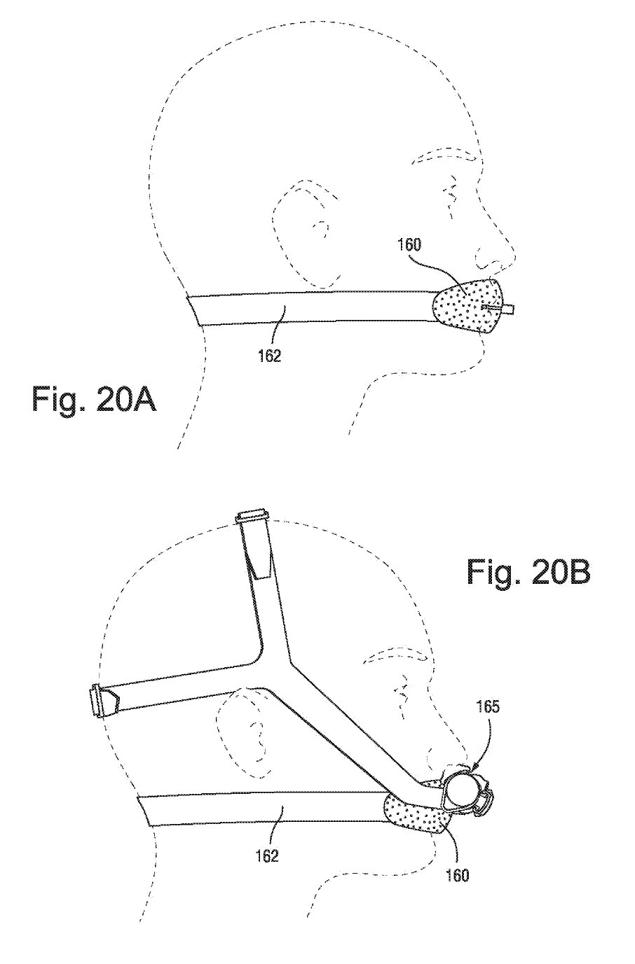

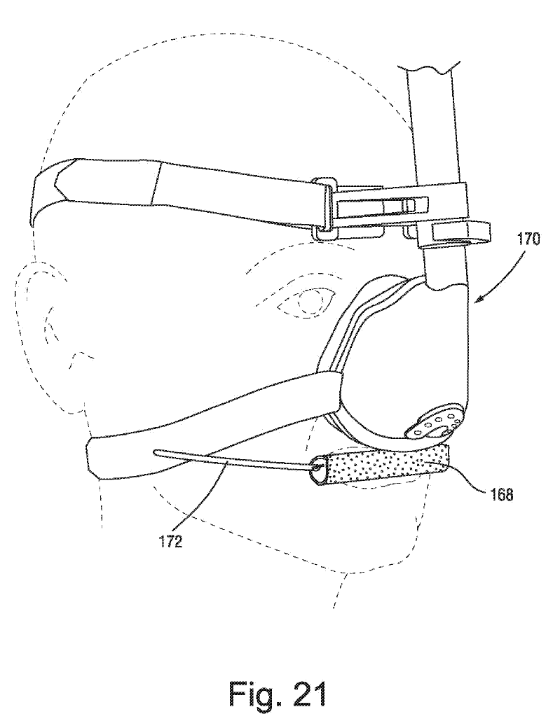

In an embodiment, a mouth seal adapted to form a seal with the patient's mouth may be constructed of a gel material, e.g., gel-filled component. FIG. 20A illustrates a mouth seal in the form of an elongated gel-filled strip 160 and a strap arrangement 162 to maintain the gel mouth seal 160 in a desired position on the patient's face, and FIG. 20B illustrates the gel mouth seal 160 being used in conjunction with a nasal prong arrangement 165. In an alternative embodiment, as shown in FIG. 21, the mouth seal may be in the form of an elongated gel tube 168. As illustrated, the gel tube 168 may be attached to a nasal mask 170 via a strap arrangement 172. Additional embodiments of mouth seals are disclosed in PCT Application No. PCT/AU2006/001246, filed Aug. 28, 2006, which is incorporated herein by reference in its entirety.

11.3 Support Structures

11.3.1 Gel Skeleton or Shell

In an embodiment, gel may be used to form a skeleton or shell that is adapted to support one or more components of a mask, e.g., cushion.

11.3.2 Gel Support Strap

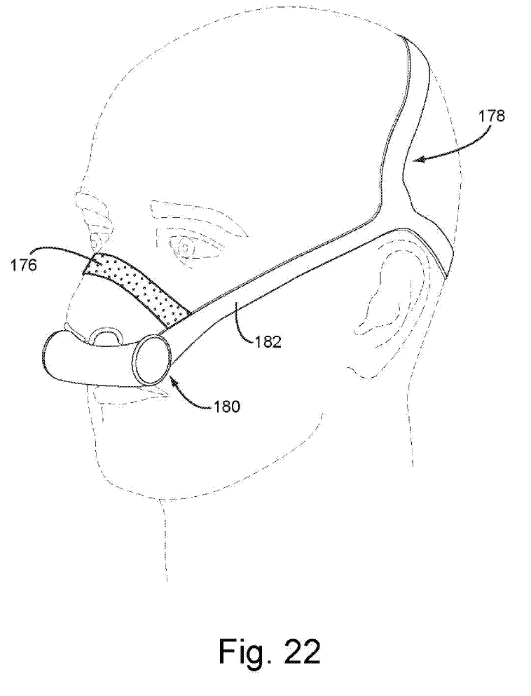

In an embodiment, gel may be used to form a strap or headgear strap of a mask. For example, as shown in FIG. 22, a gel support strap 176 may be provided to headgear 178 for a nasal prong arrangement 180 (e.g., such as the SWIFT® nasal prong arrangement described above). As illustrated, the gel support strap 176 extends between headgear side straps 182 and sits over the patient's nose to further secure the nasal prong arrangement 180 in position. In an embodiment, the gel support strap 176 may change the headgear vectors and assist in stabilizing a nasal prong arrangement while minimizing headgear requirements.

11.3.3 Gel Pads

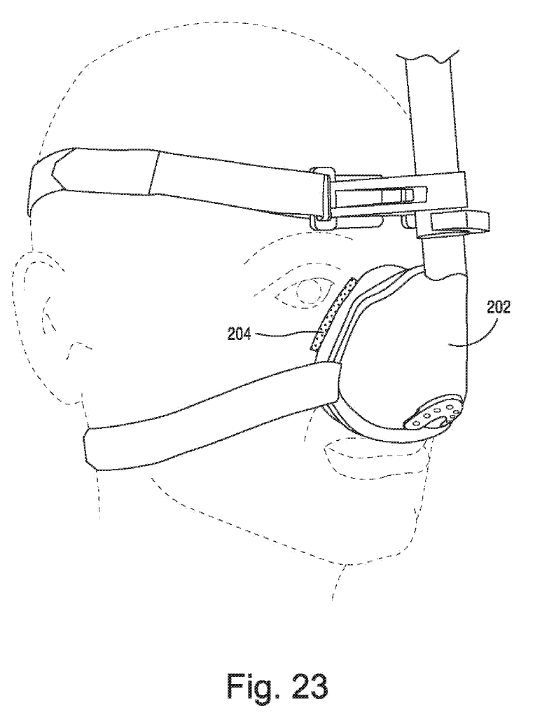

In an embodiment, gel pads may be provided to a mask that are adapted to rest on the sides of the patient's nose in use (e.g., gel pads rest on the alar sidewalls). For example, FIG. 23 illustrates a nasal mask 202 including gel pads 204 adapted to rest on the patient's nose in use.

11.3.4 Gel Earplugs

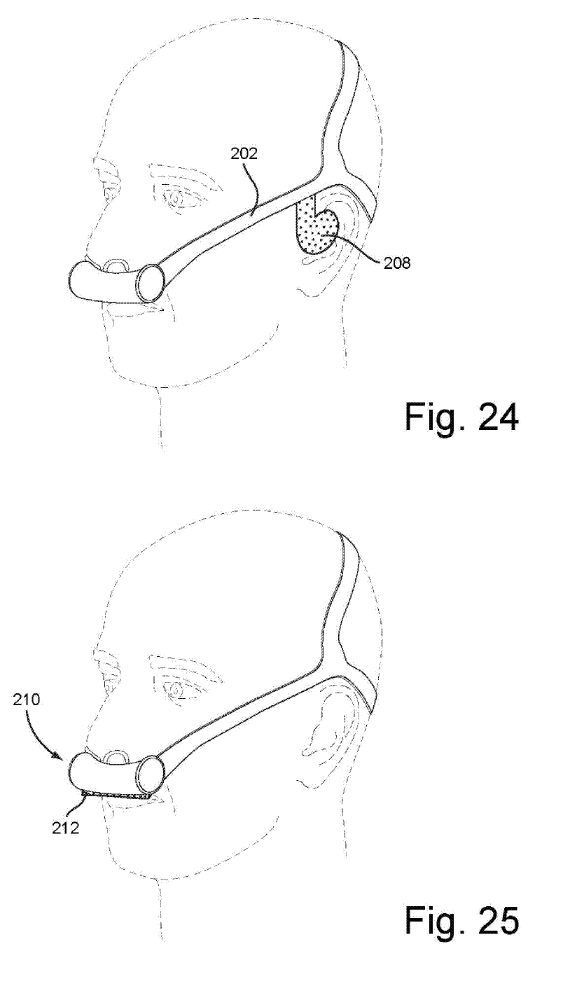

In an embodiment, gel earplugs may be provided to headgear to enhance mask retention. For example, FIG. 24 illustrates headgear 206 including gel earplugs 208 that are adapted to extend from respective side straps and engage respective ears of the patient in use. In an embodiment, the gel earplugs may be provided to the patient's inner ear and act as a mounting point to brace the headgear.

11.4 Comfort

11.4.1 Comfort Support Pad

In an embodiment, a comfort support pad may be provided that is adapted to engage the patient's upper lip in use. For example, FIG. 25 illustrates a nasal prong arrangement 210 that includes a support pad 212 adapted to engage the patient's upper lip in use.

11.4.2 Inserts

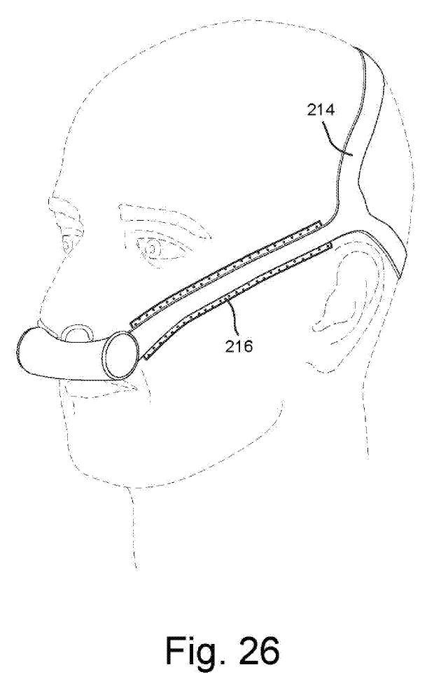

In an embodiment, inserts (e.g., gel or foam inserts) may be provided to the inner side of one or more headgear straps to prevent the headgear from making marks on the patient's face in use. For example, FIG. 26 illustrates headgear 214 including gel and/or foam inserts 216 that are provided to respective side straps and adapted to engage the patient's face in use.

11.4.3 Gel Pads

In an embodiment, gel pads may be provided to one or more portions of the mask.

11.4.4 Heated Gel

In an embodiment, gel may be provided with heat pack crystals (e.g., sodium acetate and water with a metal clicker) or other suitable heating arrangement to provide a heated gel in use.

11.4.5 Impregnated Foams

In an embodiment, foam may be impregnated with oils or scents adapted to stop the patient's skin from drying out during the night and generally provide the patient's skin with a helpful product.

11.5 Alignment and/or Stability

In an embodiment, gel may be incorporated into a mask to improve alignment and/or stability in use. For example, gel may be incorporated into the gusset portion of a gusseted cushion to act as a mechanical dampener in use.

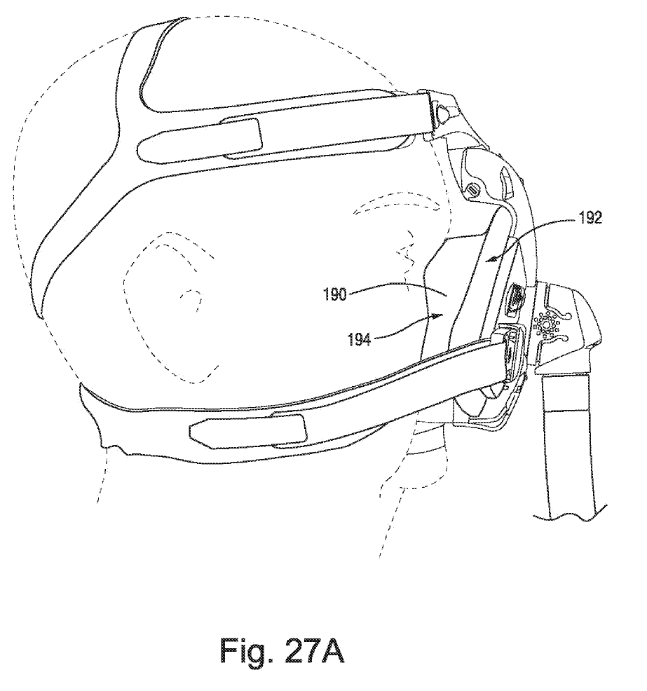

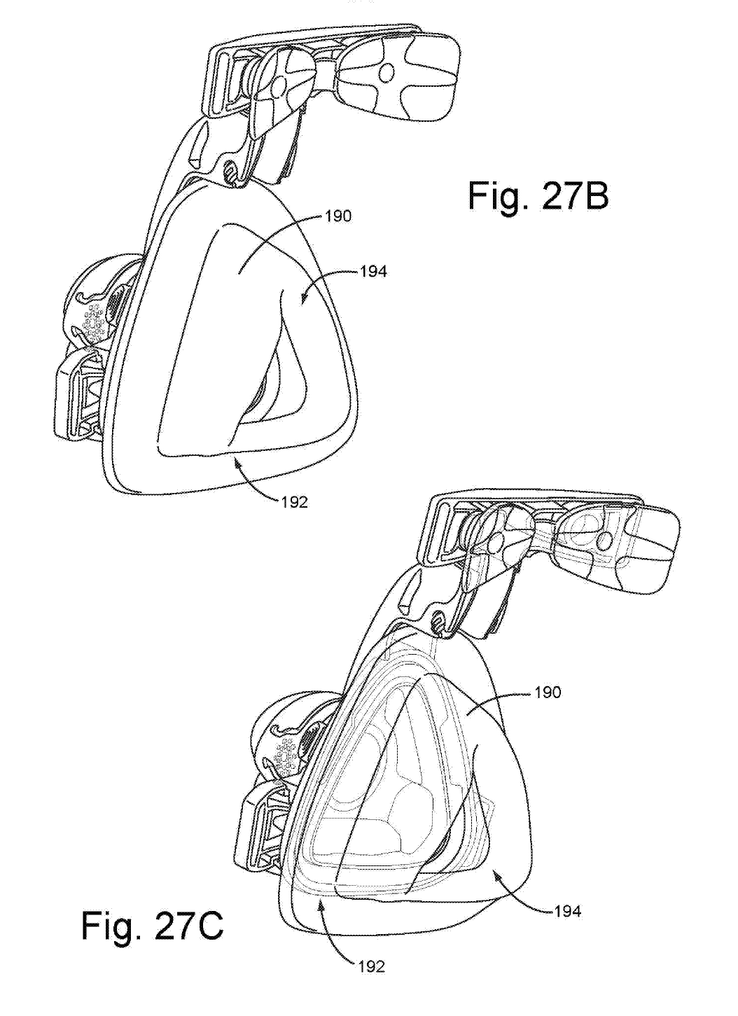

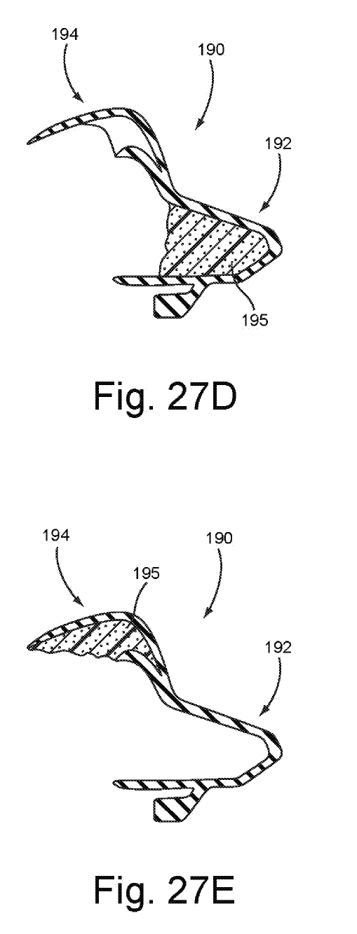

FIGS. 27A to 27E illustrate ResMed's Activa mask including a gusseted cushion 190, which is disclosed in U.S. patent application Ser. No. 10/655,622, filed Sep. 5, 2003, the entirety of which is incorporated herein by reference. In an embodiment, as shown in FIG. 27D, the gusset portion 192 of the gusseted cushion 190 may be filled with gel 195 to dampen or slow movements of the cushion in use. That is, the gel may be adapted to prevent or at least reduce the “bounce” effect of the cushion and may flatten out the movement of the mask with respect to the patient's face in use. Thus, the gel-filled gusset portion is adapted to stabilize the mask and enhance the seal. The gel-filled gusset portion may be particular useful for dampening movement when using a bi-level flow generator.

In an alternative embodiment, as shown in FIG. 27E, the seal portion 194 of the gusseted cushion 190 may be filled with gel 195 and the gusset portion 192 may be provided without gel. This arrangement would provide a gel seal onto the patient's face while the gusset portion may be used for seal enhancement.

In another embodiment, gel pads may be provided to a nasal prong arrangement (e.g., such as the SWIFT® nasal prong arrangement described above) to act as stabilizers in use.

11.6 Headgear Applications

11.6.1 Gel Pads

In an embodiment, gel pads may be provided to one or more portions of mask headgear.

11.6.2 Gel/Foam Headgear

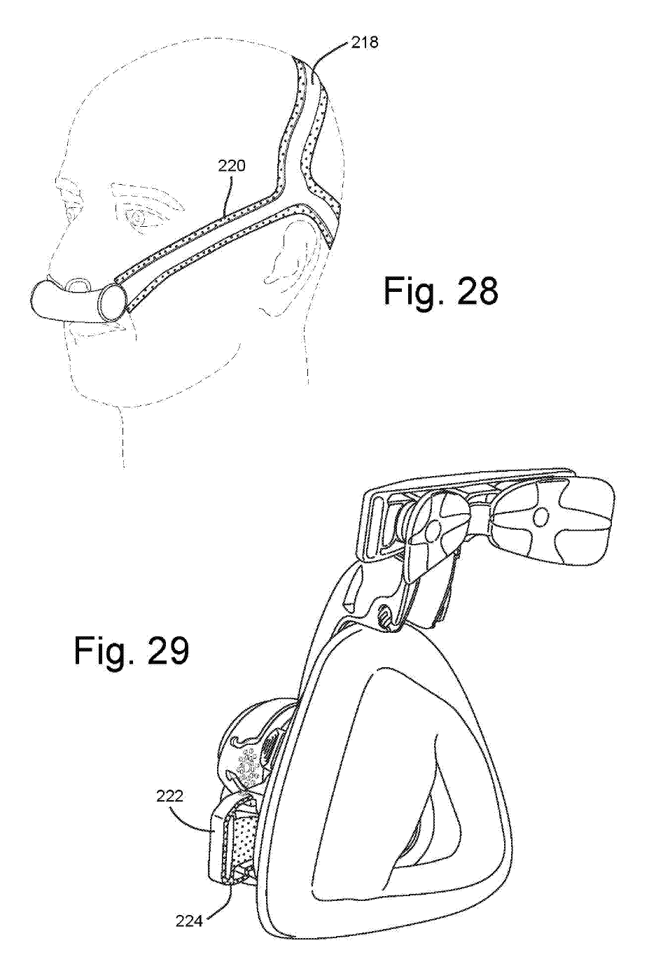

In an embodiment, gel and/or foam may be used in headgear to give it a more self-supporting structure. For example, FIG. 28 illustrates headgear 218 including gel and/or foam 220 on the inner side of the headgear straps that is adapted to engage the patient's face in use. In an embodiment, memory foams may be used that structured to conform to the contours of the patient's head and/or face.

11.6.3 Gel Pads or Memory Foam Pads

In an embodiment, gel pads or memory foam pads may be provided on the inside of buckles and/or adjustment clips of mask headgear to prevent the patient's head from resting against the relatively hard material of the buckles or clips. For example, FIG. 29 illustrates a mask including a headgear clip 222 with gel and/or foam pads 224 on the inner side thereof.

11.6.4 Gel/Foam Ear Pads

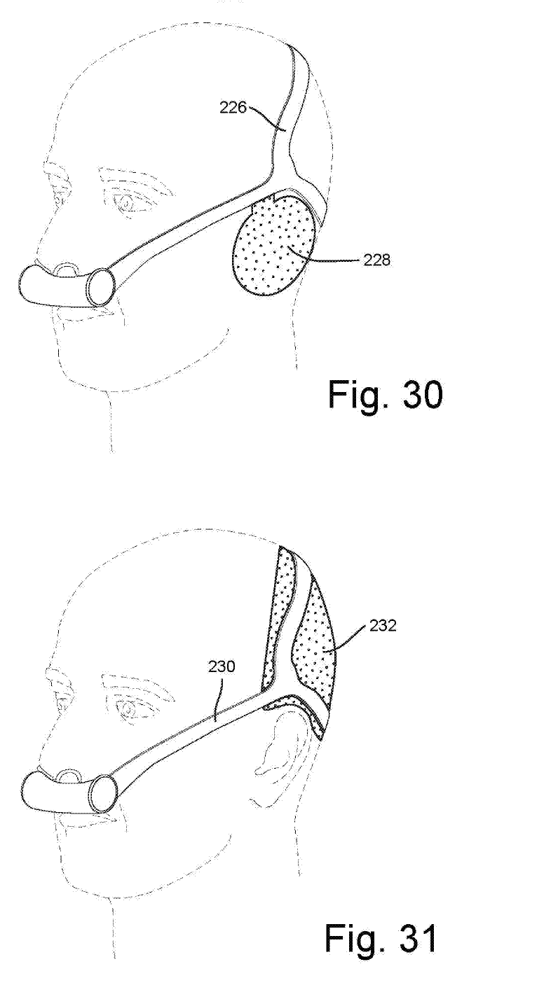

In an embodiment, gel and/or foam ear pads may be provided that are adapted to extend over the patient's ears in use (e.g., ear pads adapted to cover or rest on the patient's ears). For example, FIG. 30 illustrates headgear 226 including gel and/or foam ear pads 228 that are adapted to extend from respective side straps and cover or rest on respective ears of the patient in use.

11.6.5 Gel Displacement

In an embodiment, gel may be provided in one or more portions of the headgear and the gel's displacement may be used to displace or shift headgear over-tensioning. For example, gel may be provided to headgear in a manner as shown in FIG. 26 or 28.

11.6.6 Gel Conduit Headgear

In an embodiment, at least a portion of conduit headgear (e.g., tubing adapted to support mask on a patient's head) may include a gel component to increase comfort in use.

11.6.7 Gel Mattress

In an embodiment, a gel mattress may be incorporated into headgear to improve comfort and conformability of the headgear in use. For example, FIG. 31 illustrates headgear 230 including a gel mattress 232 provided to a rear portion thereof.

11.6.8 Additives

In an embodiment, additives (e.g., air bubble, glitter, color, air bubbles via an aeration process, glass beads, etc.) may be provided to gel and/or foam which can provide a visual indication where compression is highest in use. Air bubbles may indicate aeration of the material.

11.7 Gel Characteristics

Gel adapted for use in breathing mask applications may provide one or more of the following characteristics: noise dampening, load distributing (reducing pressure points), soft feel regions, energy conversion (e.g., heatable), tacky features, and/or clear/tintable. In an embodiment, the “flow” of gel may be adapted to provide a seal around nasal hairs like mucous. In another embodiment, the gel may be microwavable to provide warmth in use.

Some alternatives to gel for use in breathing mask applications include: oil-suspended foam, visco-elastic foam, floam, and low duro silicone.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention. Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment. In addition, while the invention has particular application to patients who suffer from OSA, it is to be appreciated that patients who suffer from other illnesses (e.g., congestive heart failure, diabetes, morbid obesity, stroke, bariatric surgery, etc.) can derive benefit from the above teachings. Moreover, the above teachings have applicability with patients and non-patients alike in non-medical applications.

What is claimed is:

1. A cushion, comprising:
a first cushion section including at least one wall member adapted to seal with a patient's face; and
a second cushion section adapted to dampen movement of the cushion in use, the second cushion section having an open configuration on at least one side thereof,
wherein the second cushion section includes at least a portion containing a gel,
wherein the first cushion section has a variable profile such that the profile of the first cushion section in a first portion of the cushion is different from the profile of the first cushion section in a second portion of the cushion.

2. The cushion according to claim 1, wherein the second cushion section extends radially outwardly.

3. The cushion according to claim 2, wherein the second cushion section extends outwardly from a side wall of the cushion and curves back inwardly into a non-face contacting portion of the cushion adapted to be attached to a frame.

4. The cushion according to claim 1, wherein the second cushion section includes a first side wall extending outwardly from a side wall of the cushion, a second side wall extending outwardly from a non-face contacting portion of the cushion adapted to be attached to a frame, and an arcuate wall that interconnects the first and second side walls.

5. The cushion according to claim 4, wherein the first side wall, the second side wall, and the arcuate wall define a space therebetween to contain the gel.

6. The cushion according to claim 1, wherein the second cushion section extends from a side wall of the cushion and curves back into a non-face contacting portion of the cushion adapted to be attached to a frame.

7. The cushion according to claim 1, wherein the second cushion section includes a first side wall extending from a side wall of the cushion, a second side wall extending from a non-face contacting portion of the cushion adapted to be attached to a frame, and an arcuate wall that interconnects the first and second side walls.

8. The cushion according to claim 7, wherein the first side wall, the second side wall, and the arcuate wall define a space therebetween to contain the gel.

9. A mask assembly, comprising:
a frame; and
a cushion according to claim 1 provided to the frame.

10. The cushion according to claim 1, wherein a height of the first cushion section in the first portion of the cushion is different than a height of the first cushion section in the second portion of the cushion.

11. The cushion according to claim 10, wherein a first thickness of the wall member in the first portion of the cushion is different than a second thickness of the wall member in the second portion of the cushion.

12. The cushion according to claim 1, wherein a first thickness of the wall member in the first portion of the cushion is different than a second thickness of the wall member in the second portion of the cushion.

13. A cushion, comprising:
a first cushion section including at least one wall member adapted to seal with a patient's face; and
a second cushion section adapted to dampen movement of the cushion in use, the second cushion section having an open configuration on at least one side thereof,
wherein the second cushion section includes at least a portion containing a gel,
wherein the gel has a variable profile such that the profile of the gel in a first portion of the cushion is different from the profile of the gel in a second portion of the cushion.

14. The cushion according to claim 13, wherein the second portion provides less support than the first portion.

15. The cushion according to claim 13, wherein the second portion has a lower height than the first portion.

16. The cushion according to claim 13, further comprising a wall that supports the gel.

17. The cushion according to claim 16, wherein the wall is configured to oppose a force generated by pressure applied to an interior portion of the cushion.

18. The cushion according to claim 13, wherein the cushion defines a perimeter that is configured to surround a user's airways and the variable profile varies around the perimeter.

19. The cushion according to claim 18, wherein a height of the profile varies around the perimeter.

20. A mask assembly, comprising:
a frame; and
a cushion according to claim 13 provided to the frame,
wherein the frame and the cushion together at least partially delimit a breathing chamber.
